(19) United States
(12) Patent Application Publication
(10) Pub. No.: US 2019/0343535 A1
(43) Pub. Date: Nov. 14, 2019

(54) METHODS AND APPARATUS FOR TREATING VEINS

(71) Applicant: Luvaura, Inc., Raleigh, NC (US)

(72) Inventor: BRUCE CHOI, Raleigh, NC (US)

(73) Assignee: Luvaura, Inc, Raleigh, NC (US)

(21) Appl. No.: 16/410,409

(22) Filed: May 13, 2019

Related U.S. Application Data
(60) Provisional application No. 62/670,452, filed on May 11, 2018.

(51) Int. Cl. A61B 17/12

(52) U.S. Cl. CPC A61B 17/12195; A61N 5/0616; A61B 2017/1205; A61B 17/12109

(57) ABSTRACT

Methods and apparatus for treating veins are disclosed. In one example, a method for treating a vein includes: positioning a distal end of a delivery tool, such as a syringe and needle or catheter, within the vein; introducing media from the delivery tool into the vein; and creating an occlusion in the vein by exposing the media to energy using an artificial energy source.

### RELATED APPLICATIONS

This application claims the priority benefit of U.S. Provisional Application No. 62/670,452, filed May 11, 2018, entitled METHOD AND APPARATUS FOR TREATING VEINS, the entirety of which is hereby incorporated by reference.

### BACKGROUND

Veins carry blood throughout the body. There are multiple types of veins, including superficial and deep, that can have various conditions that can benefit from medical treatment. For example, a condition known as telangiectasias, i.e., spider veins, involves a network of small superficial veins that become visible at the surface of the skin. Such veins form near the surface of the skin and are easily visible due to their typical deep red, blue or purple color. They may form in areas on the legs, nose, cheeks, and hands, among other places on the body. While they may cause some discomfort, telangiectasias are typically cosmetic, though they may be a sign of progressive vascular diseases, such as chronic venous insufficiency (CVI). The veins in the legs of individuals with CVI have diseased valves that normally keep blood from pooling in the legs. As a result the veins will become varicosed, leading to pain, swelling, discomfort, skin ulcers, and even death.

Current methods for varicose veins include sclerotherapy, laser or radiofrequency (RF) ablation, and adhesive based treatment. While endovascular RF or laser ablation and currently available adhesive based treatments are effective in treating varicose veins due to their larger diameter and less tortuous geometry they are not suitable for the treatment of spider veins. Thus of the primary therapies for the treatment of larger varicose veins, only sclerotherapy is feasible for the treatment of spider veins.

Sclerotherapy, which involves an injection of sclerosing chemicals that damage the inner lumen of the veins, causing them to thrombose and eventually occlude, do not require the placement of a catheter within the diseased vein to deliver therapy. Instead, the sclerosant is injected into the spider veins using a syringe and fine gauge needle where it then flows through the vein bed consisting of numerous small diameter, highly tortuous veins.

There are several shortcomings with sclerotherapy for the treatment of spider and varicose veins. Since the treatment of the diseased vein is achieved by first damaging the inner lumen of the vessel and then allowing it to heal such that the opposing walls adhere to each other, patients experience inflammation and thus discomfort following treatment. Sclerotherapy will often require multiple treatments to effectively occlude the diseased vein, which is one of the main shortcomings of the existing treatment. Physicians will often supplement sclerotherapy with the use of compression stockings to promote occlusion, but even so, multiple treatments are frequently required. Such multiple treatments are costly, inconvenient and uncomfortable for the patient. Additionally, since the sclerosant is systemically circulated throughout the human body, physicians must also consider the effects outside of the treatment zone.

Topical lasers are also utilized to treat spider veins (but not varicose veins). Such lasers are only able to treat very small vessels and also require several weeks to heal, as well as multiple treatments to achieve results. Other drawbacks include the potential for discoloring skin. Such topical lasers, thus, may only be suitable for a minority of spider vein cases.

Currently adhesive-based treatment uses strong, fast acting adhesives, i.e., cyanoacrylates. The treatment has multiple drawbacks, including not being viable for the treatment of spider veins. Instead, the treatment has been viable for conditions in larger veins, namely varicose veins. Among other disadvantages, the adhesives used in the past rapidly polymerize upon contact with blood or tissue. This makes handling and delivery to the treatment area difficult. It would also prevent proper placement and distribution of the adhesive within the network of micro-vessels typical of spider veins. Further, for varicose veins, current adhesive treatment requires the use of high viscosity formulations to displace blood. For spider veins, the high viscosity would further prevent the adhesive from adequately infiltrating the spider vein network. For varicose veins, the high viscosity formulation requires the use of a larger inner diameter of the catheter to accommodate delivery to the treatment location. As such, this places a limitation on the tortuosity of the vein that can be treated as larger diameter catheters are stiffer making them more difficult to place in such veins.

As previously mentioned, endovascular RF and laser ablation therapies are not suitable for the treatment of spider veins. Such therapies have multiple drawbacks, including the need to place a catheter in the treatment vessel. Furthermore, such ablative therapies require the use of tumescent anesthesia to protect against skin burns and pain. Application of tumescent anesthesia would not be feasible for spider veins due to their small size and network and is a drawback for thermal ablation therapies.

### BRIEF DESCRIPTION OF THE DRAWINGS

Aspects of the present disclosure are best understood from the following detailed description when read with the accompanying figures. It is noted that various features are not necessarily drawn to scale. In fact, the dimensions and geometries of the various features may be arbitrarily increased or reduced for clarity of discussion. Like reference numerals denote like features throughout specification and drawings.

FIG. 1 schematically illustrates treatment of a network of spider veins in accordance with some embodiments of the present disclosure.

FIG. 2 is a flow chart illustrating an exemplary method for treating a vein, such as the spider vein, in accordance with some embodiments of the present disclosure.

FIG. 3 illustrates an exemplary structure of an apparatus that provides an energy source for treating a vein, in accordance with one embodiment of the present disclosure.

FIG. 4 illustrates another exemplary structure of an apparatus that provides an energy source for treating a vein, in accordance with one embodiment of the present disclosure.

FIG. 5 illustrates yet another exemplary structure of an apparatus that provides an energy source for treating a vein, in accordance with one embodiment of the present disclosure.

FIG. 6 schematically illustrates an apparatus for treatment of a varicose vein in accordance with some embodiments of the present disclosure.

FIG. 7 schematically illustrates treatment of a varicose vein in accordance with some embodiments of the present disclosure.

FIG. 8 is a flow chart illustrating an exemplary method for treating a vein, such as the spider vein, in accordance with some embodiments of the present disclosure.

FIG. 9 illustrates an exemplary structure of a kit providing item for treatment of a vein, in accordance with some embodiments of the present disclosure.

### DETAILED DESCRIPTION

The following disclosure describes various exemplary embodiments for implementing different features of the subject matter. Specific examples of components and arrangements are described below to simplify the present disclosure. These are, of course, merely examples and are not intended to be limiting. For example, the formation of a first feature over or on a second feature in the description that follows may include embodiments in which the first and second features are formed in direct contact and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

Further, spatially relative terms, such as “beneath,” “below,” “lower,” “above,” “upper” and the like, may be used herein for ease of description to describe one element or feature's relationship to another element(s) or feature(s) as illustrated in the figures. The spatially relative terms are intended to encompass different orientations of the device in use or operation in addition to the orientation depicted in the figures. The apparatus may be otherwise oriented (rotated 90 degrees or at other orientations) and the spatially relative descriptors used herein may likewise be interpreted accordingly. Terms such as “attached,” “affixed,” “connected” and “interconnected,” refer to a relationship wherein structures are secured or attached to one another either directly or indirectly through intervening structures, as well as both movable or rigid attachments or relationships, unless expressly described otherwise.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

Reference will now be made in detail to the present embodiments of the disclosure, examples of which are illustrated in the accompanying drawings. Wherever possible, the same reference numbers are used in the drawings and the description to refer to the same or like parts. As appreciated by a person of ordinary skill in the art, detail expressly described with respect to one embodiment or figure should be understood to apply to, and understood as implicitly disclosed with respect to, other embodiments and figures of this disclosure.

The present teaching provides a method for treating spider veins. In one embodiment, the spider veins are treated based on a liquid embolic formulation, for example, a biocompatible epoxy based formulation that cures with exposure to ultraviolet (UV) light or other radiation. Because the curing radiation or light can be delivered through the skin, the liquid embolic can be formulated with a low viscosity, such that the liquid embolic can flow through small gauge needles and fully infiltrate the spider vein network before it is cured in place.

The disclosed method addresses short comings of sclerotherapy as a cured embolic will remain stationary within the treated spider vein network. Vein occlusion in the disclosed method will occur through acute coaptation of the treated vessels followed by a chronic occlusion of the treated vessels due to a foreign body reaction to the cured embolic (as opposed to the normal healing process initiated through sclerotherapy). This will typically permit a successful treatment of spider veins with a single treatment, while most sclerotherapy treatments require multiple injections over the course of weeks or months to occlude the vein.

By placing a curing source of energy around the telangiectasias, the disclosed method can also prevent migration of the liquid embolic outside of the treatment zone. Once cured, the embolic will no longer be able to migrate. While most epoxies that can be used as the liquid embolic are clear, it is possible to combine colorants and/or medications to help modify the color of the treated veins and further improve the aesthetics and/or healing process.

Unlike the multiple sessions required for certain prior existing treatments, including sclerotherapy, in some embodiments, the disclosed methods allow as little as a single treatment to permanently occlude the affected veins. Furthermore, unlike certain prior treatments, including current adhesive treatment, the disclosed teachings provide an ability to leverage flow through spider veins to fully infiltrate a treatment zone; an ability to deliver a liquid embolic into the tortuous and small diameter vasculature found in telangiectasias; and an ability to cure a liquid embolic once placed in the treatment zone.

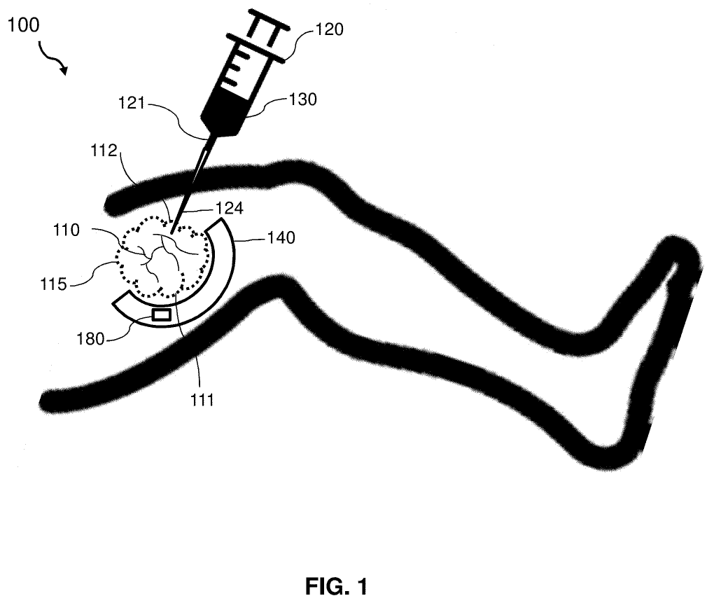

FIG. 1 schematically illustrates treatment of a network of spider veins 110 in treatment zone 115 using a system 100 in accordance with some embodiments of the disclosure. The system 100 comprises a delivery tool (e.g., syringe 120 and fine gauge needle 124), curable media 130 and energy source 140. In accordance with one embodiment, treatment comprises providing a syringe 120 with a distal end 121 for injecting, using a fine gauge needle 124, a curable media 130 into a vein of network 110.

As shown in FIG. 1, fine gauge needle 124 is introduced near the proximal end 112 of a treatment zone 115, where curable media 130 is released. In one embodiment, the curable media 130 is a liquid embolic that is designed to have appropriate viscosity to permit it to flow like blood through the treatment zone. Accordingly, proximal end 112 is preferably disposed at a location of the treatment zone that is upstream relative to the expected blood flow through the network of spider veins 110. The curable media 130 is introduced to the network of spider veins 110 via fine gauge needle 124, such that curable media 130 flows from the proximal end 112 to the distal end 11 of treatment zone 115.

A high viscosity, e.g., larger than 1000 centipoise (cP), would make curable media 130 incompatible with smaller diameter, tortuous veins such as those of spider vein network 110. In addition, such a high viscosity media would be difficult to pass through fine gauge needle 124. Further, curable media 130 is adapted not to cure until it is exposed to a specific form and amount of energy, e.g., UV light. Hence, unlike a cyanoacrylate used in the prior art, curable media 130 does not polymerize upon contact with blood or tissue. The curable media 130 therefore has the advantage that it is able to infiltrate the spider vein network 110.

Accordingly, curable media 130 preferably has a low viscosity for the treatment of small diameter or spider veins. In some embodiments, curable media 130 is a liquid embolic. In one embodiment, curable media 130 has a viscosity less than 500 cP, allowing it to migrate. In another embodiment, curable media 130 has a viscosity less than 100 cP. In yet another embodiment, curable media 130 has a viscosity of 1 cP. In yet another embodiment, the curable media 130 has a viscosity greater than 500 cP at room temperature but has a temporarily lower viscosity at the time of injection through the application of heat. In some embodiments, the curable media 130 has a viscosity appropriate for administration via small gauge needle 124, preferably having a gauge selected within the range of 21 G to 33 G.

Energy source 140 is an artificial energy source, such as an electrically or chemically powered light or radiation source, or other artificial source of energy not naturally generated by the patient's body. For example, energy source 140 may be an ultraviolet (UV) light source. As shown in FIG. 1, energy source 140 is provided at the distal end 111 of the treatment zone 115. The curable media 130 infiltrates the spider vein network 110 by virtue of natural flow mechanisms.

External pressure may also be applied at the surface of the skin to direct the curable media 130 into the spider vein network. Portions of curable media 130 become exposed to energy as it flows proximate to energy source 140. In some embodiments, upon exposure, curable media 130 cures, safely occluding the affected spider vein, preventing further migration of the curable media 130 outside of the treatment zone 115. Preferably, the energy emitted from energy source 140 causes curing of curable media 130, as opposed to curing due to interaction with blood or tissue. Acutely the occlusion may be resultant of an adhesive bond to the inner lumen of the treated vessel or due to mechanical interference with the small diameter of the tortuous spider vein. Chronically, occlusion of the treated veins are created via a foreign body response to the now solid embolic media that renders the occlusion permanent. In some embodiments, energy source 140 is adapted to be manipulated into position as an energy barrier at least partially surrounding a given treatment zone 115. Accordingly, embolic migration out of the treatment zone can be mitigated. Curable media 130 is cured at the distal end 111, which halts further migration. In some embodiment, energy source 140 comprises one or more light rings about the treatment zone.

Furthermore, since curable media 130 will not cure until exposed to the energy from energy source 140, it is possible to manipulate the location of the embolic within treatment zone 115 to ensure uniform application within the network of spider veins 110. Once curable media 130 is thoroughly implanted, the energy source 140 may be manipulated to direct energy across treatment zone 115 to cure and permanently occlude the network of spider veins 110. As such, an occlusion in the veins 110 will be created by exposing the curable media 130 to the energy emitted by the energy source. In some embodiments, energy source 140 may include more than one emitter of energy, for example, a first emitter adapted to form a barrier to migration out of the treatment zone, and a second emitter adapted to be simultaneously manipulated by a treatment professional to cure the curable media 130 throughout treatment zone 115. As between the first and second emitters in some embodiments, the energy, e.g., UV light, can differ in magnitude and type.

In some embodiments, the energy source 140 can be placed into contact with a patient's skin prior to introducing curable media 130 into the network of spider veins 110. For example, the source of energy may be directly affixed to the patient's skin using tape or the like to secure a boundary around a treatment zone 115.

In one embodiment, to mitigate the extent to which a superficial vein can be felt through the skin, curable media 130 is adapted to cure in a highly pliable solid form. In some embodiments, curable media 130 can also be formulated to cure in a porous solid form such that it will promote tissue ingrowth and rapid chronic occlusion. This can be achieved by promoting the creation of gases during the curing process or infusing the curable media with resorbable particles.

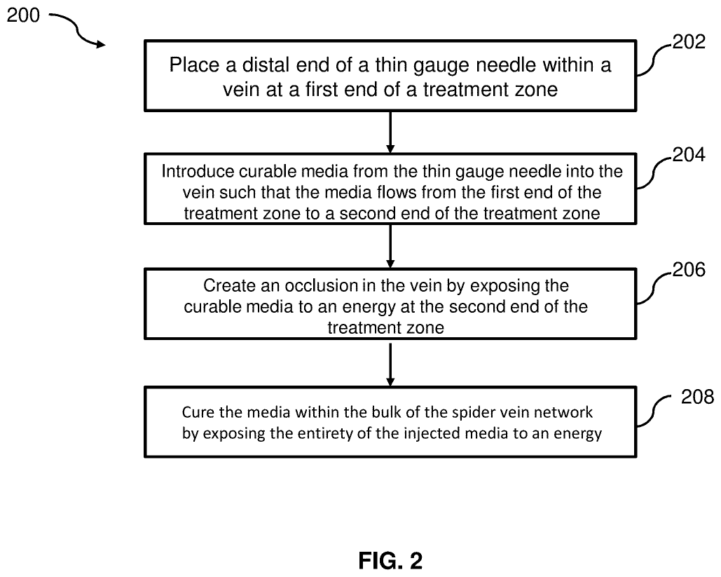

FIG. 2 is a flow chart illustrating an exemplary method 200 for treatment of a vein, such as those in the network of spider veins 110, in accordance with some embodiments of the present disclosure. At operation 202, a distal end of a thin gauge needle is placed within a vein at a first end of a treatment zone. At operation 204, curable media, e.g., a liquid embolic, is introduced from the thin gauge needle into the vein such that the media flows from the first end of the treatment zone to a second end of the treatment zone. At operation 206, an occlusion is created in the vein by exposing the curable media to an energy at the second end of the treatment zone. At operation 208, the media is cured within the bulk of the spider vein network by exposing the entirety of the injected media to an energy. In some embodiments, operation 206 and/or 208 may involve an energy source having more than one energy emitter, for example, a first emitter adapted for operation 206 to form a barrier to migration out of the treatment zone, and a second emitter adapted for operation 208 to be manipulated by a treatment professional to cure the curable media 130 throughout treatment zone 115.

The order of the operations shown in FIG. 2 may be changed according to different embodiments of the present disclosure. Further operations described in this disclosure may further be integrated with the method illustrated in FIG. 2.

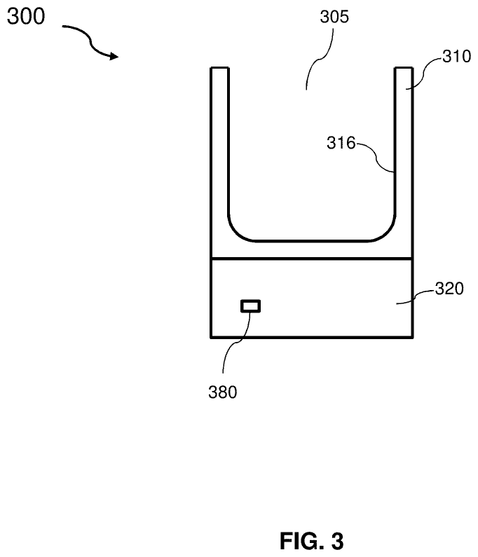

FIG. 3 illustrates an exemplary structure of an apparatus 300 that provides a light source for treating a vein, in accordance with some embodiments of the present disclosure. As shown in FIG. 3, the apparatus 300 includes a base 320, a support 310 coupled to the base 320, and an energy source 316, such as a UV light source, retained by the support 310. The energy source 316 is configured to apply an external energy into a vein such that media flowing in the vein cures to create an occlusion in the vein upon exposure to the external energy. The energy could be, for example, electrical stimulation, cryotherapy, infrared, visible, or UV light, microwave, RF radiation, other electromagnetic radiation, ultrasound energy, magnetic energy, thermal energy, nuclear or a combination of the above energy sources.

As shown in FIG. 3, the energy source 316 has a U shape with an opening 305, such that the light source 316 can be placed around a human body part, e.g. an arm or a leg. The base 320 can serve as a handle for a person to hold the apparatus 300. In addition, the base 320 may include controller 380 comprising a power source, processor and a memory for control, recording and/or processing data related to the vein treating process. For example, controller 380 is adapted to allow the user to activate all or a portion of energy source 316 as appropriate for a location, shape or size of a treatment zone. The power source of controller 380 may include, for example, a rechargeable battery or power supply circuitry to receive electric power from, e.g., a wall outlet.

In some aspects and embodiments disclosed, treatment is effected using energy such as light transmitted through the skin. Thus, the energy source, such as one or more light emitters, are preferably placed directly on the surface of the skin, in addition or alternatively to, the inner diameter of a loop. Preferably, the energy source, such as a light, is adapted to cure the bulk of the spider vein network. In some embodiments, the entirety of the skin contacting surface of the energy source structures disclosed herein are energy (e.g., light) emitting.

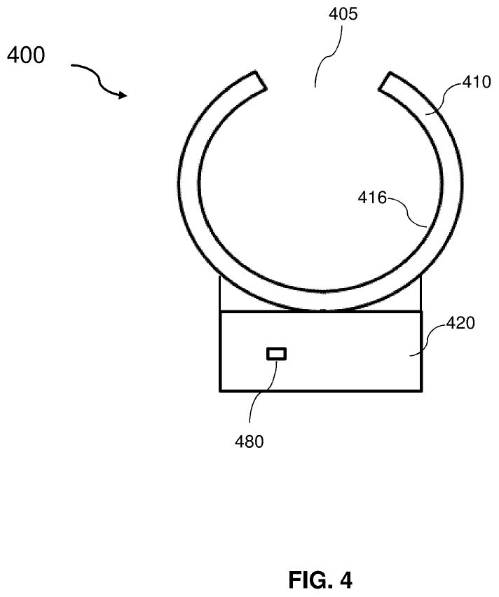

FIG. 4 illustrates another exemplary structure of an apparatus 400 that provides a light source for treating a vein, in accordance with some embodiments of the present disclosure. As shown in FIG. 4, the apparatus 400 includes a base 420, a support 410 coupled to the base 420, an energy source 416 retained by the support 410, and a controller 480. The energy source 416 is configured to apply an external energy into a vein such that media flowing in the vein cures to create an occlusion in the vein upon exposure to the external energy.

As shown in FIG. 4, the energy source 416 has an open-circle shape with an opening 405, such that the light source 416 can be placed around a human body part, e.g., an arm or a leg. Alternatively, the central axis of support 410 may be disposed orthogonal to the plane of a patient's skin, providing a light barrier. In such an orientation, opening 405 can provide an access space for a treatment professional to apply curable media to a treatment zone. The base 420 can serve as a handle for a person to hold the apparatus 400. In addition, controller 480 may include a power source, processor and a memory for controlling apparatus 400, including controlling the energy emitted, and recording and/or processing data related to the vein treating process.

In various embodiments, the light source can take on geometries suitable to the treatment site and that at least partially encircles the spider vein network. In some embodiments, the curing in the treatment will commence along the periphery of the treatment zone with a first emitter, and once cured, treatment may continue with a second emitter, preferably larger in size, to cure the central portion of the treatment zone. In some embodiments, a more targeted energy source, such as a light pencil, is used to spot cure locations.

In various aspects, the disclosed systems, methods and kits, are adapted to cure media inside the body through the application of energy. In some embodiments, energy is delivered through the skin. In some embodiments, energy is delivered from within a vein using an endovascular catheter, for example an endovascular fiber optic catheter. In some embodiments any combination of the energy sources disclosed herein may be used together, as would be understood by a person of ordinary skill in the art following the teachings of this disclosure.

In some embodiments, treatment involves curing adhesive in long varicose veins through the skin, and may include the use of a hand held wand to apply pressure and energy, while travelling down the vein. In some embodiments, treatment of long varicose veins involves curing with an endovascular energy source, such as a light source.

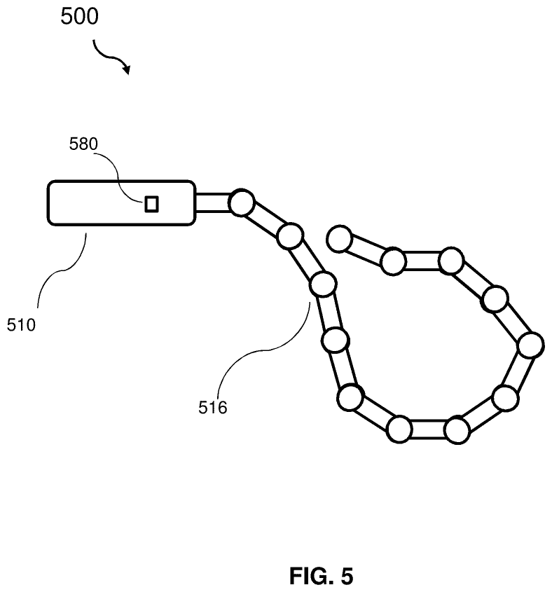

FIG. 5 illustrates yet another exemplary structure of an apparatus 500 that provides an energy source 516 for treating a vein, in accordance with some embodiments of the present disclosure. As shown in FIG. 5, the apparatus 500 includes a support 510, controller 580, and segmented energy sources 516 that may be manipulated to partially or entirely enclose the spider vein network. The energy sources 516 are configured to apply an external energy into a vein such that curable media flowing in the vein cures to create an occlusion in the vein upon exposure to the external energy.

In some embodiments, support 510 and energy sources 516 have an adhesive that can be used to affix energy source 516 temporarily about a treatment zone. In other embodiments, energy sources 516 retain their positioning such that it can be secured temporarily around a portion of a patient's body. For example, apparatus 500 can bend inward to form an open-circle with an opening or a closed circle around the treatment zone.

Apparatus 500 may also include a controller 580 comprising a power source, processor and a memory for controlling energy source 516 and recording and/or processing data related to the vein treating process. For purposes of convenience, sanitation, or cost effectiveness, apparatus 500 may be separable into two or more parts, some of which comprising an attachment designed to be disposable for limited use, such as single use for a treatment. For example, support 510 may be a disposable attachment adapted to be connected to controller 580 for the duration of a treatment session.

In accordance with various embodiments, the energy source 140 in FIG. 1 can be implemented as any one of the energy sources shown in FIGS. 3-5, and can be implemented with other shapes or structures consistent with the principles of the operation of the treatment methods taught by this disclosure. The energy emitted by the energy source 140, for example, UV light, is used to cure the curable media through the skin, which may, in some embodiments, be used to prevent migration out of a treatment zone. An energy source placed at the perimeter of the treatment area is used to ensure the curable media does not migrate outside the treatment area. Once the perimeter is cured, the energy source may be applied to the entire surface of the spider vein network to cure the media thoroughly.

Preferably, in some embodiments, the energy source is not applied at the access point (i.e. the proximal end 112 where the curable media 130 is released) to prevent curing the curable media before it could infiltrate a treatment zone.

In some embodiments, the energy source 140 is a UV light array. The array may be LEDs arranged in concentric circles. In one example, the outer ring illuminates first, then the interior LEDs illuminate progressively as curable media is confirmed to be in the treatment zone.

Energy source 140 includes controller 180 comprising a power source, processor and a memory for control, recording and/or processing data related to the vein treating process. For example, controller 180 is adapted to allow the user to activate all or a portion of energy source 140 as appropriate for a location, shape or size of a treatment zone. The power source of controller 180 may include, for example, a rechargeable battery or power supply circuitry to receive electric power from, e.g., a wall outlet.

In another embodiment, the energy source may be applied intravascularly as opposed to through the skin. This may be accomplished through the use of fiber optics to transport light energy or the placement of the energy source at the end of the catheter. This embodiment would be appropriate for treating larger diameter veins with fewer branches, such as varicose veins.

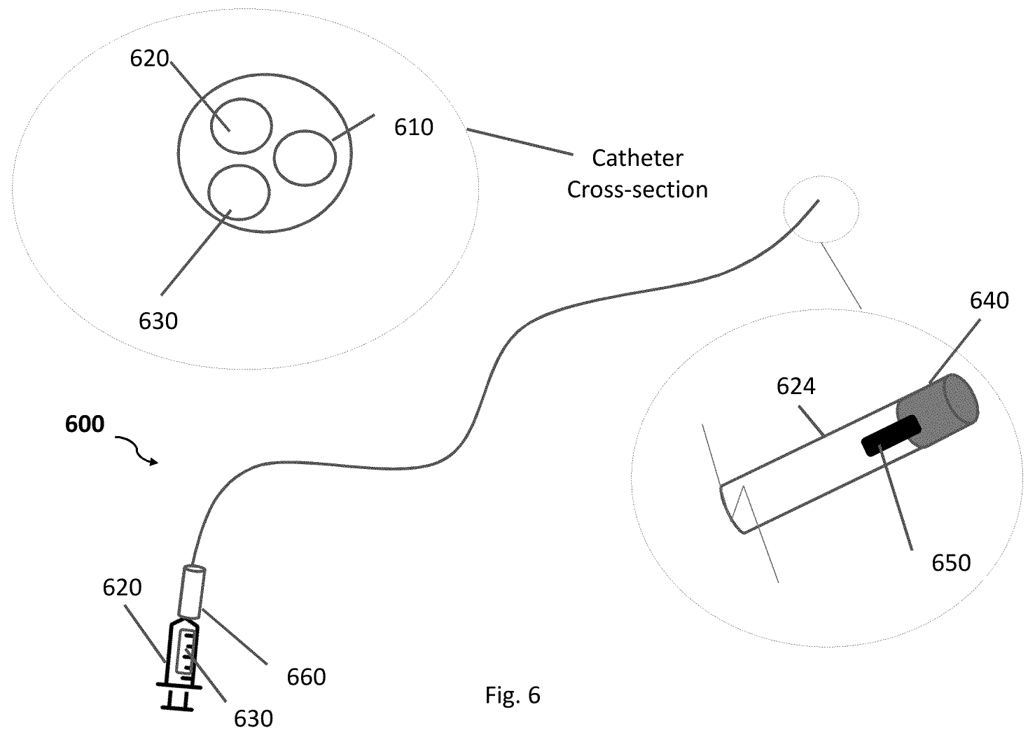

FIG. 6 illustrates an exemplary structure for a delivery tool comprising intravenous apparatus 600. Apparatus 600 has an energy source 640 at the tip of the catheter 624. The apparatus consists of a catheter 624 that serves to deliver the curable media 630 and deliver the energy source 640. Preferably, the cross section of the apparatus includes three lumen, which may be distinct. Lumen 610 serves to transport the curable media 630 from a syringe 620 or other dispenser attached to apparatus connector 660. Lumen 620 serves to pass the fiber optic or conductive wire through the catheter 624 to the illuminate or power the energy source 640. In embodiments having lumen 630, the lumen can be used to pass a guide wire through the catheter 624 to facilitate proper placement of the apparatus within the vasculature. When metered quantities of curable media 630 are dispensed through the apparatus 600, media 630 will emerge from an opening at the distal end of the catheter through opening 650. Concurrently the physician will activate energy source 640 to cure media 630 when it is properly positioned.

Cyanoacrylate liquid embolics ideally are designed with higher viscosities, e.g. larger than 1200 centipoise (cP), to displace blood that will also initiate polymerization upon contact. In doing so the initiate polymerization yields longer polymer chains capable of bonding to the vessel wall as opposed to short chain polymers that bond only to blood. This higher viscosity and rapid polymerization makes cyanoacrylate based media incompatible with smaller diameter veins such as spider veins as it would have difficulty to infuse a complex vein network. Further, the high viscosity adhesive would not be able to pass through a fine gauge needles and require larger diameter lumen for delivering into longer vessels. As such, the curable media 130 is designed to have a low viscosity to facilitate transport to the treatment location and the ability to polymerize upon demand. In contrast to cyanoacrylates, which polymerize ionically, epoxies do not. Preferably, epoxy resins bond to themselves, and accordingly, displacement of blood in epoxy based embodiments should not be required according to the teachings of the present disclosure.

In one embodiment, the curable media 130 has a viscosity less than 500 cP to allow it to migrate. In another embodiment, the curable media 130 has a viscosity less than 100 cP. In yet another embodiment, the curable media 130 has a viscosity as low as 1 cP. In yet another embodiment the curable media 130 has a high viscosity at room temperature but is temporarily lowered at the time of injection through the application of heat. In some embodiments, the curable media 130 has a viscosity to be administered with small gauge needles, including needles having a gauge selected within the range of 25 G and 33 G. However, in other embodiments, where transport of the media is more tolerant of higher viscosities (i.e. the treatment of varicose veins), the viscosities may be higher, exceeding 500 cP.

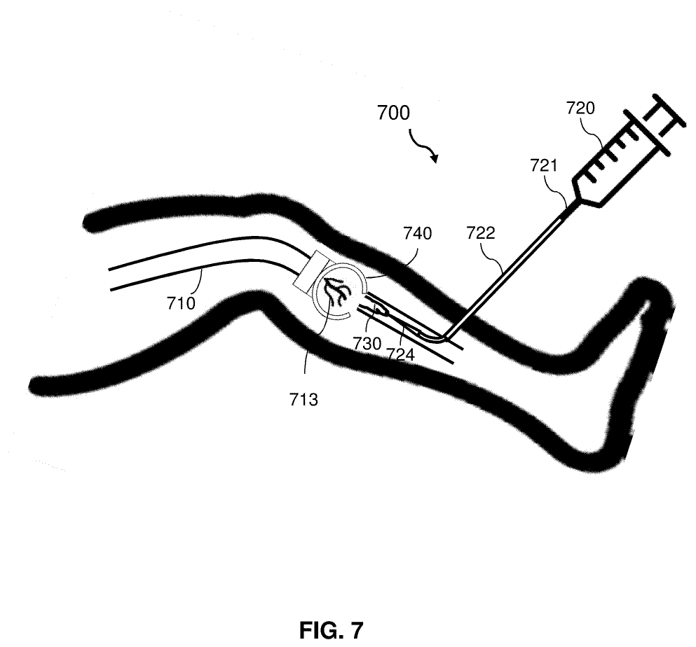

FIG. 7 schematically illustrates a method for treating a larger diameter vessel 710 having a varicose vein 713 requiring treatment. The method uses system 700 in accordance with some embodiments of the disclosure. System 700 includes syringe 720, catheter 724, curable media 730 and energy source 740. In accordance with some embodiments, the method comprises providing syringe 720 with distal end 721 for injecting curable media 730 into vessel 710. In some embodiments, syringe 720 is operably connected to catheter 724 having sheath 722 surrounding it to assist in providing access to a target site within the vessel 710 interior.

As shown in FIG. 7, catheter 724 is introduced near a treatment zone of the vessel 710 proximal to varicose vein 713. Curable media 730 is introduced from the catheter 724 into the vessel 710 and selectively cured through the application of energy either through the skin using energy source 740 or intravascularly, using, for example, apparatus 600 described with respect to FIG. 6. The viscosity of curable media 730 may be higher for larger vein treatments as compared to spider vein treatment.

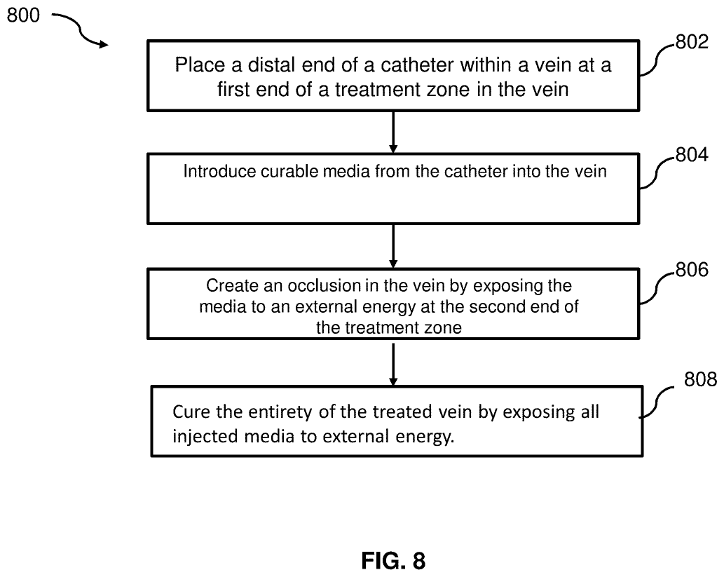

FIG. 8 is a flow chart illustrating an exemplary method 800 for treating a deep vein, such as the varicose vein, in accordance with some embodiments of the present disclosure. At operation 802, a distal end of a catheter is placed within a vein at a first end of a treatment zone in the vein. At operation 804, curable media, e.g. a liquid or foam embolic, is introduced from the catheter into the vein such that the curable media flows from the first end of the treatment zone to a second end of the treatment zone. At operation 806, an occlusion is created in the vein by exposing the media to energy at the second end of the treatment zone. At operation 808, treatment can include curing the entirety of the treated vein by exposing all injected media to external energy. The order of the operations shown in FIG. 8 may be changed according to different embodiments of the present disclosure. Furthermore, a person of ordinary skill in the art should understand that other operations described in this disclosure may be integrated into the method illustrated in FIG. 8

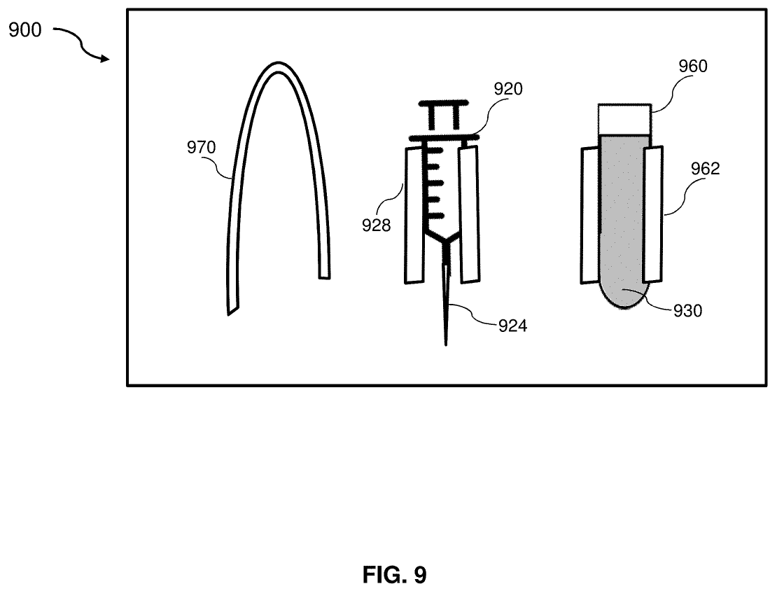

FIG. 9 shows a kit 900 for storage and/or delivery of apparatus for providing vein treatment equipment to a treatment professional. Kit 900 includes a container 960, such as a vial secured between fixtures 962. Container 960 is preferably opaque and contains curable media 930.

In some embodiments, kit 900 further includes syringe 920 and needle 924, held securely in place by fixtures 928. Alternatively, the syringe 920 may be opaque and further adapted to store curable media 930 while in kit 900. Kit 900 may further include at least a portion of an energy source. For example, kit 900 may include attachment 970 designed for use with a separable controller. In other embodiments, kit 900 may include the controller and entire energy source. The kit 900 is preferably opaque and tightly sealed to prevent or mitigate inadvertent exposure to a curing form of energy while curable media 930 is in storage, transit or otherwise awaiting use by a treating professional.

In some embodiments, curable media 930 is a liquid embolic. In others, it may be a foam to treat larger veins. Curable media 930 can also be combined with dyes, pigments, or pharmaceuticals to aid in cosmetic outcomes or healing. Additives can be included in the curable media 930.

In various aspects, the present specification teaches systems, apparatus, kits and methods for treating a vein. For example, systems, apparatus and kits are disclosed that are adapted to implement a method of treatment, where the method includes positioning a distal end of a delivery tool, such as a syringe and catheter or needle, within the vein; introducing media from the delivery tool into the vein; and creating an occlusion in the vein by exposing the media to an artificial energy source to create the occlusion, for example by curing the media. In some embodiments, the delivery tool introduces said media at a first end of a treatment zone such that the media flows from the first end of the treatment zone to a second end of the treatment zone and the artificial energy source is disposed at the second end of the treatment zone to create the occlusion. In some embodiments, the artificial energy source is external to a skin of a body having the vein. In other embodiments, the artificial energy source is disposed internal to the vein.

In some embodiments, the treatment zone may be manipulated to ensure uniform application of the media in the venal network of the treatment zone, prior to the media curing. In some embodiments, the vein is permanently occluded as a result of the media being cured due to exposure to the energy.

As a further example, a kit for treating a vein is disclosed. The kit includes: at least a portion of an artificial energy source, such as a sterile portion. In some embodiments the portion of the artificial energy source that is in the kit is disposable. In some embodiments, the kit includes the entirety of the artificial energy source. The artificial energy source is adapted to generate a first type of energy. The kit further includes a container encasing media, the container adapted to shield the media from the first type of energy, the media having the ability to be introduced through the distal end of a delivery tool (such as a needle or catheter) into a vein, and create an occlusion in the vein upon exposure to the first type of energy from the artificial energy source, whereby the container prevents exposure of the media to the first type of energy in storage. In some embodiments, the media and delivery tool are adapted for the media to move, for example flow, from a first end of a treatment the zone to a second end of the treatment zone and the energy source is adapted to be disposed at the second end of the treatment zone. In some embodiments, the artificial energy source is disposed external to a skin of a body having the vein, and delivers energy through the skin to the vein to create the occlusion, for example by curing the media. In some embodiments, the delivery tool comprises a catheter and the artificial energy source is positioned internal to the vein and delivers energy from within the vein to cure the media to create the occlusion. In some embodiments, the container is an opaque vial. In some embodiments, the kit includes a syringe, which may preferably be opaque. In some embodiments, the container is a syringe. In some embodiments, the kit includes at least part of the delivery tool, such as a syringe.

As a further example, an apparatus for treating a vein is disclosed. The apparatus includes: a support and an artificial energy source coupled to the support. The artificial energy source is adapted to apply an energy to media in the vein to create an occlusion in the vein upon exposure to the energy, for example, by curing the media. In some embodiments, the media is adapted to move, such as by flowing, from a first end of a treatment zone to a second end of the treatment zone and the artificial energy source is adapted to be disposed at the second end of the treatment zone. In some embodiments, the artificial energy source is adapted to apply the energy from external to a skin of a body having the vein. In others, the delivery tool includes a catheter and the artificial energy source is adapted to apply the energy intravenously from within the vein.

In some embodiments, the energy is electromagnetic energy, including one of gamma rays, x rays, ultraviolet radiation, visible light, infrared radiation, microwaves and radio waves. In some embodiments the energy is light and the artificial energy source is a light source. In some embodiments, the light source is a source of ultra-violet radiation. In some embodiments, the light source may be configured to be positioned external to the body during treatment. Alternatively, the artificial energy source may be adapted to be deployed internal to the body, including intravenously, to deliver the light to the affected vein from a location internal to the body.

In some embodiments the artificial energy source may be adapted to form a barrier of energy to limit a distribution of at least a portion of the media to non-affected areas of the venous system. In some embodiments, the barrier formed by the source is adjustable by changing the operation and/or guidance of radiating elements on the source, including for example light emitting diodes. In some embodiments, the artificial energy source is disposable. In some embodiments, the artificial energy source includes one or more flexible emitting structures placed proximal to the affected vein, to at least partially surround the area of treatment.

In some embodiments, the media is an adhesive. In some embodiments the media is a liquid embolic formulation. In some, a foam. In some embodiments the media is a biocompatible epoxy. In some embodiments, when cured, the media forms a pliable solid such that it cannot be felt through the patient's skin and will not fracture with movement or the application of external forces. In some embodiments, the media forms a porous solid when cured, whereby tissue ingrowth and chronic occlusion is improved. In some embodiments the media has a viscosity substantially acceptable for a sclerosant. In some embodiments the media has a viscosity low enough to permit flow through a 25 gauge needle. In some embodiments, the media has a viscosity low enough to permit flow through a 33 gauge needle. In some embodiments the viscosity of the media is less than 500 cP. In some embodiments, it is less than 100 cP. In others, it is 1 cP. In yet another embodiment, the curable media 130 has a viscosity greater than 500 cP at room temperature but has a temporarily lower viscosity at the time of injection through the application of heat. In some embodiments, the adhesive is non-cyanoacrylate. In some embodiments, the media is clear. In some embodiments the media includes a colorant. In some embodiments the media has a medicament.

In some embodiments, the vein is a spider vein. In some embodiments, the vein is a varicose vein. In some embodiments, the disclosed treatment techniques avoid the need to use compression during the treatment. Preferably, the disclosed treatment techniques avoid or reduce the need for the patient to wear compression items, such as compression socks or bands, after treatment.

In some embodiments for treating varicose veins, the energy source is intravenously delivered to the treatment area, for example, in conjunction with use of a catheter. In some embodiment the curing energy source is located adjacent to the distal end of the catheter or needle from which the curable media 130 is dispensed. This may be achieved by the placement of the energy source emitter, or, for the embodiment utilizing UV light as the energy source, a fiber optic cable can be placed along the catheter. In some intravenous embodiments, the catheter and energy sources are biocompatible and incapable of forming a bond with the occluding media.

In some embodiments, the affected vein is treated in a single patient treatment on an outpatient basis. In some embodiments, the treatment leverages flow through the affected spider veins to cause the media to infiltrate the spider vein. In some embodiments the treatment is able to deliver the media into the tortuous and small diameter vasculature of telangiectasias. In some curing embodiments, the treatment involves delaying the curing of the media, preferably liquid embolic, until after the media infiltrates the treatment area. In some embodiments, the disclosed treatment techniques avoid or mitigate the need for compression during or after treatment, for example when treating spider veins.

The foregoing outlines features of several embodiments so that those ordinary skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

What is claimed is:

1. A method for treating a vein, comprising:
positioning a distal end of a delivery tool within the vein proximate a treatment zone in the vein;
introducing media from the delivery tool into the vein; and
creating an occlusion in the vein by exposing the media to energy using an artificial energy source.

2. The method of claim 1, wherein said step of introducing media comprises introducing said media into the vein at a first end of said treatment zone and said step of creating an occlusion occurs proximate to a second end of said treatment zone after said media flows through said treatment zone.

3. The method of claim 1, wherein the artificial energy source is positioned external to a skin of a body having said vein, and delivers energy through the skin to said vein to create said occlusion.

4. The method of claim 1, wherein the delivery tool includes a catheter and the method further comprises the steps of positioning said artificial energy source internal to the vein and delivering energy from within said vein to create said occlusion.

5. The method of claim 1, wherein said media comprises a liquid embolic having a viscosity less than 500 cP during said step of introducing media into the vein, and wherein the creating an occlusion step includes curing said liquid embolic.

6. The method of claim 1, wherein said energy is ultraviolet light.

7. The method of claim 1, further comprising positioning said artificial energy source to form an energy barrier to limit distribution of at least a portion of said media to said treatment zone.

8. A kit for treating a vein, comprising:
at least a portion of an artificial energy source, wherein the artificial energy source is adapted to generate a first type of energy;
a container encasing media and adapted to shield said media from said first type of energy;
wherein the media is adapted to be introduced through a distal end of a delivery tool into the vein and create an occlusion in the vein upon exposure to said first type of energy from said artificial energy source.

9. The kit of claim 8, wherein said at least a portion of said artificial energy source is disposable.

10. The kit of claim 8, wherein said artificial energy source is adapted to be positioned external to a skin of a body having said vein, and to deliver energy through said skin to said vein to create said occlusion.

11. The kit of claim 8, wherein said delivery tool comprises a catheter and said artificial energy source is adapted to be positioned internal to said vein and to deliver energy from within said vein to create said occlusion.

12. The kit of claim 8, wherein said media comprises a liquid embolic formulated to have a viscosity less than 500 cP when introduced into said vein and to cure when exposed to said first type of energy.

13. The kit of claim 8, wherein said first type of energy is ultraviolet light.

14. An apparatus, comprising:
a support; and
an artificial energy source coupled to said support, wherein the artificial energy source is configured to apply an energy to a media in a vein in a treatment zone such that said media in the vein creates an occlusion in the vein upon exposure to the energy.

15. The apparatus of claim 14, wherein said media is adapted to flow from a first end of said treatment zone to a second end of said treatment zone and said artificial energy source is adapted to expose said media proximate said second end of said treatment zone.

16. The apparatus of claim 14, wherein said artificial energy source is adapted to be positioned external to a skin of a body having said vein, and to deliver energy through said skin to said vein to create said occlusion.

17. The apparatus of claim 14, wherein said delivery tool comprises a catheter and said artificial energy source is adapted to be positioned internal to said vein and to deliver energy from within said vein to create said occlusion.

18. The apparatus of claim 14, wherein said media comprises a liquid embolic formulated to have a viscosity less than 500 cP when introduced into said vein and to cure when exposed to said energy.

19. The apparatus of claim 14, wherein said energy is ultraviolet light.

20. The apparatus of claim 14, wherein said support is adapted for positioning said artificial energy source to form an energy barrier to limit distribution of at least a portion of said media to said treatment zone.
